(19) United States
(12) Patent Application Publication
(10) Pub. No.: US 2021/0145904 A1
(43) Pub. Date: May 20, 2021

(54) Use of Lactobacillus Plantarum S58 in Preparation of Product for Alleviating Spicy Food-Induced Damage to Digestive System

(71) Applicant: Southwest University, Chongqing (CN)

(72) Inventor: Huayi Suo, Chongqing City (CN)

(73) Assignee: Southwest University, Chongqing (CN)

(21) Appl. No.: 16/950,323

(22) Filed: Nov. 17, 2020

(30) Foreign Application Priority Data
Nov. 19, 2019 (CN) 201911131496.5

(51) Int. Cl. A61K 35/747; A61K 9/00; A61P 1/14; A61K 47/36

(52) U.S. Cl. CPC A61K 35/747; A61K 47/36; A61P 1/14; A61K 9/0056

(57) ABSTRACT

The disclosure provides use of Lactobacillus plantarum S58 with an accession number of CCTCC NO: M 2019595 in the preparation of a health food and a medicament for alleviating spicy food-induced damage to the digestive system. The disclosure not only expands the application range of the L. plantarum S58 and improve the utilization value thereof, but also brings new hope for the treatment of patients with digestive ulcer caused by frequent consumption of chilli.

### TECHNICAL FIELD

The disclosure relates to use of Lactobacillus plantarum in the preparation of a health product and a medicament.

### BACKGROUND

With the rapid popularity of Sichuan cuisine and Chongqing hot pot at home and abroad, there are more eaters. Spicy taste in hot pot comes from capsaicin in chili. Capsaicin is an oxamide-containing alkaloid with a molecular formula of C₁₈H₂₇NO₃, and chemical name thereof is trans-8-methyl-N-vanillyl-6-nonenamide. Studies have shown that proper capsaicin intake has anti-tumor, anti-analgesic, anti-inflammatory, fat oxidation, and weight reduction effects, but excessive capsaicin intake can cause damage to the human digestive system and even digestive ulcers. Therefore, the ultimate effect of capsaicin depends on the dose administered.

In the past decade, according to incomplete estimates, digestive system diseases accounted for 42% of all diseases, including digestive ulcer, ulcerative colitis, some chronic cholecystitis and post-hepatitis syndrome. The digestive system including the digestive tract (mouth, larynx, esophagus, stomach, small intestine, and colon) and auxiliary digestive organs (pancreas, gallbladder, and liver) produces different reactions due to the above-mentioned conditions, and ultimately manifests as a digestive disease. About 10% of people in the world will suffer from digestive ulcers every year. Digestive ulcers have become one of the most important gastrointestinal diseases in the world because of increasingly high morbidity and mortality thereof; unlike infectious diseases, digestive system ulcers are multifactorial, and the main causes are unhealthy lifestyles and different risk factors, such as physicochemical or biological injuries. As the first barriers of the digestive system against external disturbances, the stomach and intestines are the most susceptible to diet and drugs. Therefore, in the case of avoiding unnecessary burdens on the body by taking drugs, it is particularly important to find an edible and medicinal substance to protect the digestive system.

Lactic acid bacteria are closely related to human health and have functions of maintaining the micro-ecological balance of intestinal flora, protecting the gastrointestinal mucosal barrier, strengthening the body's immune function, preventing and inhibiting tumorigenesis, improving the utilization of food nutrients, promoting the absorption of nutrients in food, and lowering cholesterol, delaying body aging, preventing dental caries, inhibiting the growth of pathogenic bacteria, etc. Unique biological characteristics and probiotic function make lactobacilli have broad application prospects and utilization value in the fields of agriculture, food and medical care.

### SUMMARY

An objective of the disclosure is to provide a L. plantarum strain isolated from pickles, and develop food, medicaments and functional health products by means of a role thereof in alleviating the digestive tract ulcer.

After research, the disclosure provides the following technical solutions:

The disclosure provides use of L. plantarum S58, which is used in the preparation of a medicament for treating or preventing spicy food-induced damage to the digestive system, where the L. plantarum S58 is deposited with an accession number of CCTCC NO: M 2019595.

The disclosure provides use of L. plantarum S58, which is used in the preparation of a food for alleviating spicy food-induced damage to the digestive system, where the L. plantarum S58 is deposited with an accession number of CCTCC NO: M 2019595.

The disclosure provides use of L. plantarum S58, which is used in the preparation of a health product for alleviating spicy food-induced damage to the digestive system, where the L. plantarum S58 is deposited with an accession number of CCTCC NO: M 2019595.

The disclosure further provides a pharmaceutical composition for treating or preventing alleviating spicy food-induced damage to the digestive system, where the pharmaceutical composition contains a pharmaceutically effective dose of L. plantarum S58 with an accession number of CCTCC NO: M 2019595.

The disclosure further provides a food for alleviating spicy food-induced damage to the digestive system. The food contains the L. plantarum S58 with an accession number of CCTCC NO: M 2019595.

The disclosure further provides a health product for alleviating spicy food-induced damage to the digestive system. The health product contains the L. plantarum S58 with an accession number of CCTCC NO: M 2019595.

The disclosure further provides a food additive for alleviating spicy food-induced damage to the digestive system. The food additive contains the L. plantarum S58 with an accession number of CCTCC NO: M 2019595.

Capsaicin-induced digestive system ulcer model mice show that: L. plantarum S58 can effectively inhibit the area of gastric ulcer; can partly alleviate the damage of gastric mucosal surface structure, make glands arranged more orderly, and infiltrate fewer epithelial cells, reduce the necrosis in the glandular cavity, reduce inflammation and lymphocyte dissolution; can keep most of the small intestinal villi to maintain a normal shape, and make inflammatory cell infiltration and edema disappear; can reduce serum levels of motilin (MTL), substance P (SP), interleukin-1β (IL-1β), tumor necrosis factor-α (TNF-α), interferon-γ (IFN-γ), lipopolysaccharide (LPS), myeloperoxidase (MPO), and soluble intercellular adhesion molecule-1 (sICAM-1), increase somatostatin (SS) level, and relieve inflammation; can up-regulate the expression of epidermal growth factor (EGF), epidermal growth factor receptor (EGFR), vascular endothelial growth factor (VEGF) genes in the mouse gastric tissue, promote the healing of ulcers, mediate the self-repair effect of epithelial cells, and reduce gastric acid secretion; can down-regulate the expression of nuclear factor kappa-B (NF-κB), TNF-α, and IL-1β in gastric and small intestine tissues, up-regulate the expression of inhibitor kappa B-alpha (IκB-α), and reduce the gastrointestinal tract inflammatory response; can down-regulate the expression of inducible nitric oxide synthase (iNOS) in gastric tissues and up-regulate the expression of endothelial nitric oxide synthase (eNOS), thereby inhibiting the inflammatory response, protecting the gastric mucosa, and inhibiting gastric ulcers; can significantly up-regulate the expression of Zonula occludens protein 1 (ZO-1), and repair the intestinal mucosal barrier. Therefore, L. plantarum S58 can partly alleviate gastrointestinal ulcers caused by capsaicin.

The disclosure has the following beneficial effects: the disclosure provides use of L. plantarum S58 (accession number: CCTCC NO: M 2019595) in the preparation of a health food and a medicament for alleviating spicy food-induced damage to the digestive system, not only expanding the scope of application of L. plantarum S58 and improving utilization value thereof, but also bringing new hope to the treatment of digestive system diseases.

Deposit of Biological Material

China Center for Type Culture Collection (CCTCC); Address: Wuhan University, Wuhan, China; Deposit Date: Aug. 1, 2019; Accession Number: CCTCC NO: M 2019595; Taxonomic Name: L. plantarum S58.

### BRIEF DESCRIPTION OF THE DRAWINGS

FIG. 1 illustrates the colony morphology (a) and Gram's staining results (b) of isolated strains.

FIG. 2 illustrates API 50 CH reaction results of L. plantarum S58.

FIG. 3 illustrates gastric tissues.

FIG. 4 is the histopathological observation of the stomach.

FIG. 5 is the histopathological observation of the small intestine.

FIG. 6 is the mRNA expression of EGF, EGFR, and VEGF in the gastric tissue.

FIG. 7 illustrates the mRNA expression of NF-κB, IκB-α, TNF-α, and IL-1β in the gastric tissue.

FIG. 8 illustrates the mRNA expression of iNOS and eNOS in the gastric tissue.

FIG. 9 illustrates the mRNA expression of NF-κB, IκB-α, TNF-α, and IL-β in the small intestine tissue.

FIG. 10 illustrates the mRNA expression of ZO-1 and Occludin in the small intestine.

In the above figures, there is no significant difference between groups marked with the same lowercase English letters (a, b, and c) (p>0.05); there is a significant difference between groups marked with different lowercase English letters (a, b, and c) (p<0.05).

### DETAILED DESCRIPTION

In order to make the objectives, technical solutions and advantages of the disclosure clearer, the preferred examples of the disclosure will be described in detail below with reference to the accompanying drawings.

I. Isolation and Identification of L. plantarum S58

1 Experimental Materials

Chongqing farmhouse naturally fermented pickles.

2 Experimental Methods

2.1 Isolation and Purification of Lactobacilli

Pickle water was collected and diluted under a 10-fold gradient, and then diluted to 10⁻⁷ successively. Four proper dilutions (dilutions of 10⁻⁴, 10⁻⁵, 10⁻⁶, and 10⁻⁷) were selected and 100 μL each of the pickle water of these concentrations was spread on an MRS solid plate, respectively. After incubating for 48 h at 37° C., single colonies with different shapes were selected, and strains were isolated using the streak plate method. The above steps were repeated until a purified strain was obtained, and the morphology was observed by Gram's staining.

2.2 PCR Amplification of 16S rDNA Sequence

Bacterial Genomic DNA Extraction Kit was used to extract the DNA of the purified strain. PCR amplification was performed using a 25 μL reaction system: template DNA 1 μL, upstream primer (10 μM) 1 μL, downstream primer (10 μM) 1 μL, 2×Taq PCR Master Mix 12.5 μL, and making up to 25 μL with sterile ultrapure water. PCR amplification conditions: initial denaturation at 94° C. for 5 min; 30 cycles of denaturation at 94° C. for 1.5 min, annealing at 55° C. for 1 min, and extension at 72° C. for 1.5 min; final extension at 72° C. for 10 min. Finally, BGI Tech Solutions Co., Ltd. was entrusted to conduct bidirectional sequencing on PCR products that passed the test; sequencing results were analyzed for homology by the BLAST program in NCBI.

2.3 Identification by API Kit

Isolated strains were cultured for 18 h at 37° C., and bacterial cells were collected by centrifugation for 15 min at 3,000 r/min. The bacterial cells were washed with sterile normal saline and resuspended as a bacterial suspension. Operations were carried out with reference to the instructions of API Kit.

3 Results and Analysis

3.1 Colony Morphology and Cell Morphology of Isolated Strains

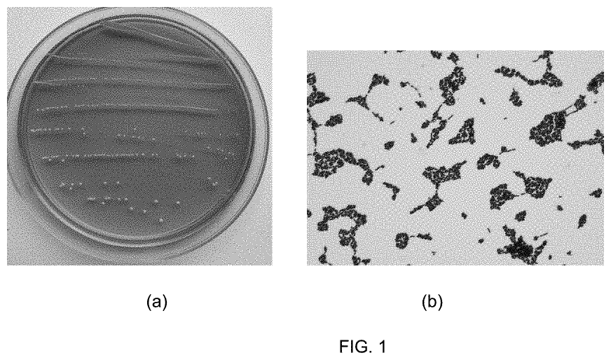

Purified strains formed single colonies in an MRS medium. The colonies had almost the same morphology, most of which appeared round, white, and smooth and moist on the surface. After Gram's staining, purple cell morphology was observed microscopically and was determined to be Gram-positive (G⁺). The colony morphology and Gram's staining result of the strains are shown in FIG. 1.

3.2 Strain 16S rDNA Sequence Analysis

The results of 16S rDNA homology analysis showed that the homology with L. plantarum known in the Gene Bank database reached 100%. A bidirectional sequence of a 16S rDNA gene amplification product of L. plantarum S58 is shown in SEQ ID No. 1.

3.3 Identification Results of Strain Biochemical Characteristics

The phenotypic identification at the Lactobacillus species level is mainly based on carbohydrate fermentation tests. API 50 CH Kit is to identify the strain's utilization of 49 different carbohydrates.

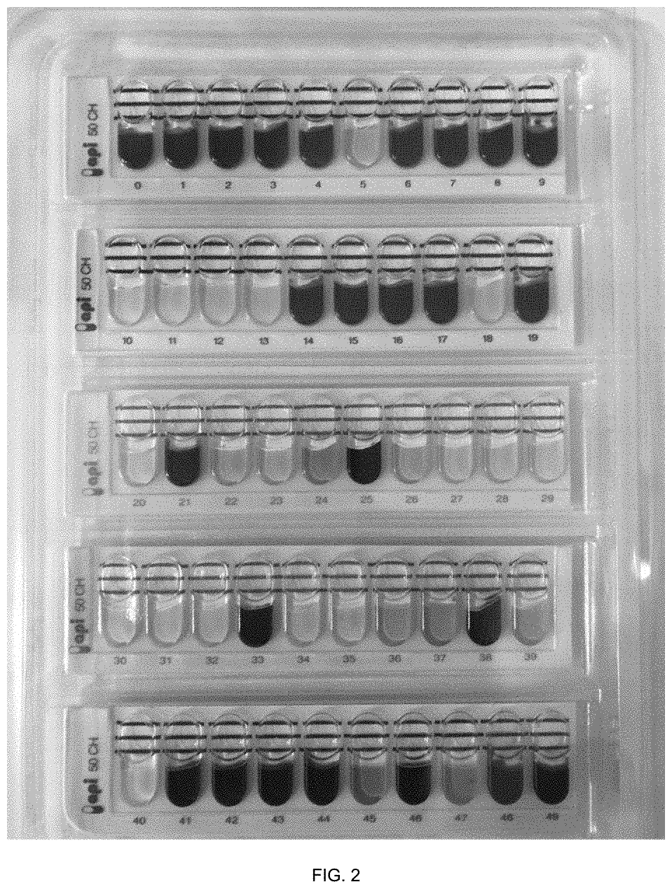

FIG. 2 illustrates the API 50 CH reaction results of the experimental strain. Table 1 shows the results of the fermentation test of the strain for 49 carbohydrates. From FIG. 2 and Table 1, of the 49 carbon sources tested, the strain can utilize 26 of these carbohydrates. After final identification by the API lab plus system, the experimental strain was L. plantarum, with an ID value of 99.90% and a T value of 0.65, which meets the identification requirements (ID value 99.0% and T value 0.5).

TABLE 1
Results of the fermentation test of
L. plantarum S58 for 49 carbohydrates

| Tube | | Reaction | Tube | | Reaction |
|---|---|---|---|---|---|
| No. | Carbohydrate | result | No. | Carbohydrate | result |
| 0 | Blank | − | 25 | Esculin and | + |
| | | | | ferric citrate | |
| 1 | Glycerol | − | 26 | Salicin | + |
| 2 | Erythritol | − | 27 | D-cellobiose | + |
| 3 | D-arabinose | − | 28 | D-maltose | + |
| 4 | L-arabinose | − | 29 | D-lactose | + |
| 5 | D-ribose | + | 30 | D-melibiose | + |
| 6 | D-xylose | − | 31 | D-sucrose | + |
| 7 | L-xylose | − | 32 | D-trehalose | + |
| 8 | D-adonitol 1 | − | 33 | Inulin | − |
| 9 | Methyl β-D- | − | 34 | D-melezitose | + |
| | xylopyranoside | | | | |
| 10 | D-galactose | + | 35 | D-raffinose | + |
| 11 | D-glucose | + | 36 | Starch | + |
| 12 | D-fructose | + | 37 | Glycogen | + |
| 13 | D-mannose | + | 38 | Xylitol | − |
| 14 | L-sorbose | − | 39 | D-gentiobiose | + |
| 15 | L-rhamnose | − | 40 | D-Toulon sugar | + |
| 16 | Dulcitol | − | 41 | D-lyxose | − |
| 17 | Inositol | − | 42 | D-tagatose | − |
| 18 | Mannitol | + | 43 | D-fucose | − |
| 19 | Sorbitol | − | 44 | L-fucose | − |
| 20 | Methyl-α-D- | + | 45 | D-arbaitol | + |
| | mannopyranoside | | | | |
| 21 | Methyl-α-D- | − | 46 | L-arbaitol | − |
| | glucopyranoside | | | | |
| 22 | N-acetyl- | + | 47 | Potassium | + |
| | glucosamine | | | gluconate | |
| 23 | Amygdalin | + | 48 | 2-Keto-potas- | − |
| | | | | sium gluconate | |
| 24 | Arbulin | + | 49 | 5-Keto-potas- | − |
| | | | | sium gluconate | |

NOTE:
“+” represents a positive reaction;
“−” represents a negative reaction.

II. Alleviating Effect of L. plantarum S58 Combined with Highland Barley β-Glucan on Digestive Ulcer in Mice

1 Experimental Materials

Natural capsaicin (purity 95%), purchased from Henan Beite Biological Technology Co., Ltd.

The experimental strain was L. plantarum S58, with an accession number of CCTCC No: M 2019595.

Highland barley β-glucan, (purity 71.2%), purchased from Xi'an Tongze Biological Technology Co., Ltd.

Experimental animals were 6-week-old male Kunming mice purchased from Chongqing Ensiweier Biotechnology Co., Ltd. They were housed in a standardized laboratory at a room temperature of 25±2° C. and a relative humidity of 50±5% on a 12 h light/12 h dark cycle. The mice were acclimatized for one week before the start of the experiment.

2 Experimental Methods

2.1 Grouping and Treatment of Experimental Animals

Fifty adult male Kunming mice were randomly divided into five groups of 10 mice. The mice free access to food and water ad libitum during modeling. The modeling and administration methods are as follows (because natural capsaicin is not readily soluble in water but readily soluble in organic solvents, soybean oil is selected as a solvent for dissolving capsaicin):

TABLE 2
Animal modeling and administration methods

| | | | Gavage | Treatment |
|---|---|---|---|---|
| Group | Mice/group | Treatment | dose | time |
| Normal | 10 | Administration of soybean oil | 0.1 ml/10 g | 4 Weeks |
| control (NC) | | by gavage in the morning | | |
| | | Administration of normal saline | | |
| | | by gavage in the afternoon | | |
| Model (CAP) | 10 | Administration of 20 mg/kg | 0.1 ml/10 g | 4 Weeks |
| | | capsaicin by gavage in the | | |
| | | morning | | |
| | | Administration of normal saline | | |
| | | by gavage in the afternoon | | |
| β-Glucan (β- | 10 | Administration of 20 mg/kg | 0.1 ml/10 g | 4 Weeks |
| D) | | capsaicin by gavage in the | | |
| | | morning | | |
| | | Administration of 300 mg/kg β- | | |
| | | glucan by gavage in the | | |
| | | afternoon | | |
| L. plantarum | 10 | Administration of 20 mg/kg | 0.1 ml/10 g | 4 Weeks |
| (LP.S58) | | capsaicin by gavage in the | | |
| | | morning | | |
| | | Administration of 1.0 × 10⁹ | | |
| | | CFU/mL bacterial suspension | | |
| | | by gavage in the afternoon | | |
| β-Glucan + | 10 | Administration of 20 mg/kg | 0.1 ml/10 g | 4 Weeks |
| L. plantarum | | capsaicin by gavage in the | | |
| (LP.S58 + β- | | morning | | |
| D) | | Administration of 300 mg/kg β- | | |
| | | glucan + 1.0 × 10⁹ CFU/mL | | |
| | | bacterial suspension by | | |
| | | gavage in the afternoon | | |

During the experiment, each mouse was weighed every three days and the amount of gavage was adjusted. At the end of week 4, feces of each group of mice were collected. All mice were deprived of food but not water for 18 h. Blood was collected by eyeball enucleation and centrifuged for 10 min at 3,000 r/m in and 4° C. to collect serum, and the serum was stored at −80° C. for use. After blood collection, the mice were sacrificed by cervical dislocation, gastric and small intestine tissues were dissected out, the gastric tissue was cut off along the greater curvature of the stomach, spread out, and quickly photographed; an appropriate amount of gastric and small intestine tissues were cut and immediately fixed in 10% formalin solution for 48 h; then all of the tissues were frozen in liquid nitrogen and finally stored at −80° C.

2.2 Tissue Section Observation

Well-fixed tissues were dehydrated, permeabilized, waxed, embedded, and sectioned for HE staining. Finally, the changes in tissue morphology were observed under an optical microscope.

2.3 Determination of Serum Markers

Levels of MTL, SP, SS, vasoactive intestinal peptide (VIP), IL-6, IL-1β, TNF-α, IFN-γ, LPS, MPO, and sICAM-1 in mouse serum were determined according to the instructions of the kit.

2.4 Determination of mRNA Expression in Gastric and Small Intestine Tissues by qPCR

Colon total RNA was extracted according to the instructions of Trizol (Invitrogen, Carlsbad, Calif., USA), 1 μL of RNA sample was mixed with 1 μL of (oligo) primer dT and 10 μL of sterile ultrapure water, and the mixture was reacted for 5 min at 65° C.; after the reaction was completed, 1 μL of Ribolock RNase Inhibitor, 2 μL of 100 mM dNTP Mix, 4 μL of 5×Reaction buffer, and 1 μL of Revert Aid M-mu/v RT were added to the reaction system. After mixing well, cDNA was synthesized at 42° C. for 60 min and at 70° C. for 5 min. The purity and concentration of the total DNA was measured by an ultramicrospectrophotometer, and then the DNA concentration of each sample was adjusted to the same level (1 μg/μL), followed by reverse transcription and amplification of target genes with the primer sequences described in Table 2. The reaction conditions were: 40 cycles of denaturation at 95° C. for 15 min, annealing at 60° C. for 1 h, and extension at 95° C. for 15 min; finally using DADPH as a housekeeping gene, the relative expression of the target genes were calculated by 2^{ΔΔCT}.

TABLE 3
Primer sequences used in the experiment

| Target gene | | Primer sequence |
|---|---|---|
| GAPDH | F | GAGGTCAATGAAGGGGTCGTT |
| | R | CTCGTCCCGTAGACAAAATGGT |
| EGF | F | TGGGTCTCGGATTGGGCT |
| | R | ACCACAACCAGTGACGAGGG |
| EGFR | F | GCCATCTGGGCCAAAGATACC |
| | R | GTCTTCGCATGAATAGGCCAAT |
| VEGF | F | TCGTCCAACTTCTGGGCTCTT |
| | R | CCTTCTCTTCCTCCCCTCTCTTC |
| NF-κB | F | ATGGCAGACGATGATCCCTAC |
| | R | CGGAATCGAAATCCCCTCTGTT |
| IκB-α | F | TGAAGGACGAGGAGTACGAGC |
| | R | TGCAGGAACGAGTCTCCGT |
| TNF-α | F | CAGGCGGTGCCTATGTCTC |
| | R | CGATCACCCCGAAGTTCAGTAG |
| IL-1β | F | GAAATGCCACCTTTTGACAGTG |
| | R | TGGATGCTCTCATCAGGACAG |
| iNOS | F | GTTCTCAGCCCAACAATACAAGA |
| | R | GTGGACGGGTCGATGTCAC |
| eNOS | F | TCAGCCATCACAGTGTTCCC |
| | R | ATAGCCCGCATAGCGTATCAG |
| ZO-1 | F | GCCGCTAAGAGCACAGCAA |
| | R | GCCCTCCTTTTAACACATCAGA |
| Occludin | F | TGAAAGTCCACCTCCTTACAGA |
| | R | CCGGATAAAAAGAGTACGCTGG |

3 Experimental Results and Analysis

3.1 Effect of L. plantarum S58 Combined with highland Barley β-Glucan on Morphology of Mouse Gastric Tissues

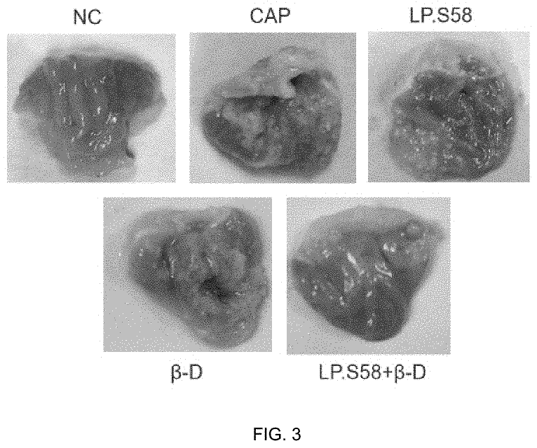

Gastric ulcers were visually observed from photos of the gastric tissue. As shown in FIG. 3, the surface of the gastric mucosa of mice in the NC group appears good, there is no obvious mucosal erosion, and the color of the stomach is bright and glossy; the mice in the CAP group have severe gastric mucosal erosion, the integrity is destroyed, and the submucosa is hyperemic and dull. The area of gastric ulcer is effectively suppressed in the β-D group, LP.S58 group, and LP.S58+β-D group from the apparent gastric ulcer area; compared with the control group, the gastric ulcer area is significantly reduced, indicating that LP .S58 has a certain protective effect on gastric ulcer caused by capsaicin, and exhibits better efficacy when combined with β-D.

3.2 Effects of L. plantarum S58 Combined with Highland Barley β-Glucan on Pathological Morphology of Mouse Gastric Tissues

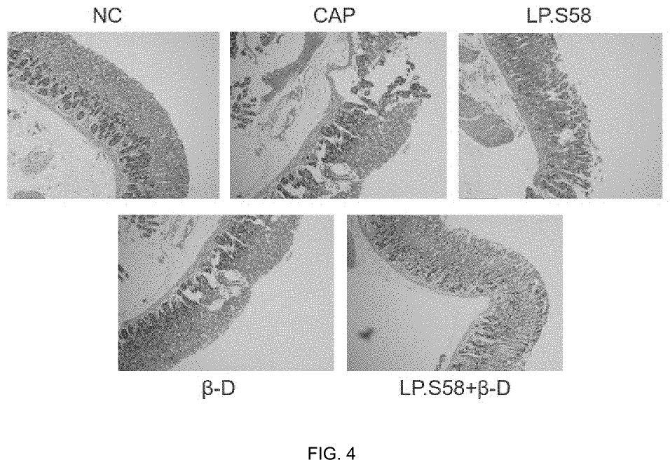

Stomach sections are another way to intuitively express the degree of capsaicin damage to gastric tissues in addition to the pictures of the stomach. As can be seen from FIG. 4, the four-layer structure of the stomach wall is clear in normal mice, whose deep epithelial structure is intact and continuous, the gland structure is neatly arranged, and gastric mucosal epidermal cells are intact, without cell infiltration and inflammation. In the CAP group, the surface structure of the gastric mucosa is severely damaged and deep into the muscular layer, where the gap is not covered by regenerating mucosa. The cell bodies are arranged disorderly with cystic dilation. The gland structure is disordered. Epithelial cell infiltration, inflammation, and lysed lymphocytes are observed. Necrotic cells appear in the glandular cavity. LP.S58 and β-D partly alleviate the damage of the gastric mucosal surface structure. The glands are arranged in an orderly manner, the epithelial cell infiltration decreases, a small amount of necrotic cells are present in the glandular cavity, and inflammation and lysed lymphocytes decrease. Particularly, the prevention effect of the LP.S58+β-D group is better than that of the LP.S58 and β-D groups, indicating that LP.S58 can partly protect gastric mucosa tissues destroyed by capsaicin.

3.3 Effect of L. plantarum S58 Combined with Highland Barley β-Glucan on Pathological Morphology of Mouse Small Intestine Tissues

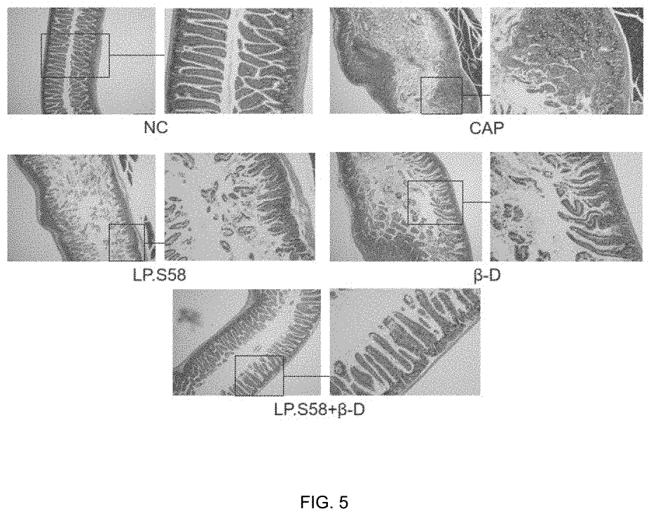

The integrity of the small intestine villi is closely related to the ability of intestinal peristalsis. Administration of capsaicin by gavage can easily cause the degradation of the intestinal villi, and even cause the intestinal villi to break and shrink. As shown in FIG. 5, the small intestinal villi of the mice of the NC group are intact and neatly arranged, and the thickness of the small intestine wall is moderate; small intestines of the mice of the CAP group are severely damaged, their villi are broken and absent and became sparse, and the small intestine wall become significantly thin, with severe inflammatory cell infiltration and edema in small intestine tissues; administration of LP.S58 or β-D alone does not significantly improve the damage to the small intestine, but the combination of LP.S58 with β-D improves the damage to the small intestine; in the LP.S58+β-D group, the vast majority of the small intestinal villi maintain normal morphology, inflammatory cell infiltration disappears, and edema subsides.

3.4 Effect of L. plantarum S58 Combined with Highland Barley β-Glucan on Serum Markers MTL, SP, SS and VIP in Mice

When the gastric mucosal barrier is damaged, highly corrosive gastric acid and pepsin can cause serious damage to the gastric mucosa and tissues. Gastrointestinal hormones are important influencing factors for regulating the secretion of gastric juice. MTL and SP are excitatory gastrointestinal hormones, the content of which will increase under stress, causing massive gastric juice secretion to enable strong acidity in the stomach, and leading to damage to the gastric mucosa tissue. SS and VIP are inhibitory gastrointestinal hormones that can inhibit the gastric juice secretion. Particularly, SS can protect the gastric mucosa by inhibiting the release of MTL and pepsin, so as to promote the healing of gastric injury. Compared with the NC group, the serum levels of MTL and SP significantly increase, and the SS level significantly decreases in the CAP group. Compared with the CAP group, the serum levels of MTL and SP significantly decrease in the LP.S58 group and the LP.S58+β-D group, and there is a more significant decrease in the LP.S58+β-D group; the serum SS level increases significantly in the LP.S58 group and the LP.S58+β-D group, and there is a more significant increase in the LP.S58+β-D group; there is no significant difference in VIP level among all groups.

TABLE 4
Effects of L. plantarum S58 combined with highland
barley β-glucan on gastrointestinal hormones in mice

| Group | MTL (ng/mL) | SP (ng/mL) | SS (ng/L) | VIP (ng/L) |
|---|---|---|---|---|
| NC | 89.74 ± 6.29^{c} | 99.43 ± 11.03^{c} | 5.46 ± 0.67^{a} | 40.48 ± 4.23^{a} |
| CAP | 120.97 ± 12.78^{a} | 167.36 ± 16.16^{a} | 3.33 ± 0.70^{c} | 42.82 ± 3.21^{a} |
| LP.S58 | 100.03 ± 8.61^{b} | 139.12 ± 22.36^{b} | 4.17 ± 0.56^{b} | 43.22 ± 1.72^{a} |
| β-D | 102.80 ± 12.29^{b} | 131,13 ± 19.44^{b} | 4.54 ± 0.62^{b} | 42.28 ± 3.85^{a} |
| LP.S58 + β-D | 96.42 ± 13.04^{bc} | 96.48 ± 11.59^{c} | 5.47 ± 0.48^{a} | 40.77 ± 4.15^{a} |

3.5 Effect of L. plantarum S58 Combined with Highland Barley β-Glucan on Serum Markers IL-6, IL-1β, TNF-α and IFN-γ in Mice

The amount of pro-inflammatory cytokines produced by inflammatory response is related to the severity of digestive ulcer and may further aggravate cell and organ damage. IL-1β is mainly produced by mononuclear macrophages, which can cause intestinal inflammation and local complications. Studies have shown that IL-6, as a chemokine, enables chemotaxis of a variety of monocytes and inflammatory cells, promote the production and release of inflammatory mediators, destroy the mucosal barrier of the digestive tract, and aggravate the inflammatory damage of the digestive tract mucosa. The expression of IFN-γ is significantly up-regulated in the ulcerous tissue, and IFN-γ is also positively correlated with the apoptosis of mucosal cells. TNF-α, which plays a synergistic role with IFN-γ, also has a variety of pathophysiological effects on digestive ulcer, including promoting apoptosis, neutrophil infiltration, causing cytoskeletal disintegration, and activation of proline and tyrosine kinase; TNF-α can also promote the release of oxygen free radicals and other pro-inflammatory cytokines, and lead to organ damage and destruction of cell membrane stability, resulting in gastrointestinal tissue damage. In this experiment, it is found that the levels of IL-1β, TNF-α and IFN-γ are the highest in the CAP group and the lowest in the NC group; the levels of IL-1β, TNF-α and IFN-γ significantly decrease in the LP.S58 group, β-D group, and LP.S58+β-D group compared to the CAP group; however, there is no significant difference in IL-6 level among groups. LP.S58 and β-D can reduce the production of cytokines and inflammatory factors to prevent digestive ulcers.

TABLE 5
Effects of L. plantarum S58 combined with highland
barley β-glucan on inflammatory factors in mice

| Group | IL-6 (ng/L) | IL-1β (ng/L) | TNF-α (ng/L) | FN-γ (ng/L) |
|---|---|---|---|---|
| NC | 58.67 ± 7.94^{a} | 57.11 ± 4.47^{c} | 305.11 ± 43.77^{c} | 736.39 ± 175.68^{b} |
| CAP | 66.17 ± 15.05^{a} | 70.55 ± 6.86^{a} | 438.91 ± 53.86^{a} | 978.06 ± 113.37^{a} |
| LP.S58 | 65.42 ± 10.21^{a} | 65.15 ± 5.09^{b} | 415.96 ± 77.45^{a} | 846.23 ± 141.32^{b} |
| β-D | 64.67 ± 5.40^{a} | 61,44 ± 4.46^{bc} | 390.26 ± 61.98^{ab} | 784.94 ± 128.66^{b} |
| LP.S58 + β-D | 63.02 ± 13.60^{a} | 57.95 ± 6.59^{c} | 340.95 ± 48.55^{bc} | 742.36 ± 103.78^{b} |

3.6 Effects of L. plantarum S58 combined with highland barley β-Glucan on Serum Markers LPS, MPO and sICAM-1 in Mice

Pathogenic intestinal flora stimulates the production and release of LPS from the intestinal epithelial cells. At the same time, intestinal flora disturbance leads to damaged intestinal mucosal barrier and increased intestinal permeability, so that LPS can easily pass through the intestinal mucosa into the bloodstream; then LPS can bind to cytokine receptors, thereby triggering the release of pro-inflammatory cytokines. MPO can activate NF-κB signaling pathway and the like, increase cytokines and adhesion molecules, make leukocytes penetrate the endothelial barrier more easily to reach inflammatory tissues, and promote the inflammatory response. Intercellular adhesion molecule sICAM-1, which can bind to specific receptors, can affect the adhesion between white blood cells and endothelial cells, enhance its adhesion, activate endothelial cells, and make it easier to penetrate the endothelial barrier and transfer to the site of injury and inflammation. As shown in Table 6, administration of capsaicin by gavage significantly increases the levels of LPS, MPO, and sICAM-1. Administration of LP.S58, β-D, or both can significantly lower 333 the serum levels of LPS, MPO, and sICAM-1, and administration of both is more effective.

TABLE 6
Effects of L. plantarum S58 combined with highland barley
β-glucan on levels of LPS, MPO and sICAM-1 in mice

| Group | LPS (ng/L) | MPO (ng/L) | SICAM-1 (ng/L) |
|---|---|---|---|
| NC | 122.37 ± 5.63^{c} | 21.14 ± 4.28^{d} | 220.03 ± 24.63^{c} |
| CAP | 166.54 ± 20.72^{a} | 30.10 ± 2.59^{a} | 265.16 ± 17.46^{a} |
| LPS58 | 139.99 ± 11.20^{b} | 26.59 ± 2.26^{b} | 244.90 ± 19.57^{b} |
| β-D | 134.19 ± 12.11^{b} | 25.49 ± 3.17^{bc} | 236.58 ± 16.11^{bc} |
| LP.S58 + β-D | 124.87 ± 11.47^{c} | 23.64 ± 2.51^{cd} | 227.75 ± 18.51^{bc} |

3.7 Effect of L. plantarum S58 Combined with Highland Barley β-Glucan on mRNA Expression of EGF, EGFR and VEGF in the Mouse Gastric Tissue

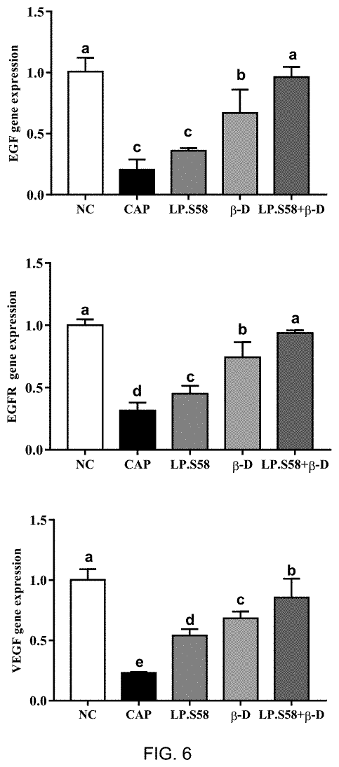

The main function of EGF is to: promote cell proliferation; participate in the proliferation and differentiation of gastrointestinal epithelial cells; regulate the growth and development of the gastrointestinal tract; protect the gastrointestinal tract; promote the migration of mucosal cells into a new granulation tissue; repair the glandular structure of a site of mucosal damage; and release a plurality of protective factors. Studies have found that EGF can inhibit the secretion of gastric acid and pepsin, and promote the synthesis of RNA- and DNA-mediated proteins, thereby protecting the gastric mucosa. VEGF can induce vascular endothelial proliferation and migration, increase vascular permeability, and play an important role in the reconstruction and neovascularization. Studies have shown that VEGF can promote the proliferation and differentiation of gastrointestinal mucosal epithelial cells, significantly increase gastric mucosal blood flow, and maintain the intestinal integrity. EGFR can mediate EGF-related effects and is widely present in mammalian and human gastrointestinal tracts. EGFR can mediate transforming growth factors around the ulcerous tissue, promote the healing of ulcer, mediate the self-repair effect of epithelial cells, and reduce the gastric acid secretion. FIG. 6 illustrates the effects of LP.S58 and β-D on the expression of EGF, EGFR and VEGF in the stomach. As seen from the figure, compared with the NC group, expression levels of EGF, EGFR and VEGF in the gastric tissue of the CAP group are significantly down-regulated; expression levels of EGF, EGFR and VEGF are partly up-regulated in both LP.S58 and β-D treatment groups, and administration of both can make the expression of EGF, EGFR and VEGF reach the level of the NC group.

3.8 Effect of L. plantarum S58 Combined with Highland Barley β-Glucan on mRNA Expression of NF-κB, TNF-α and IL-1β in the Mouse Gastric Tissue

Among the pro-inflammatory cytokines, NF-κB is an important signal transcription factor in inflammation and is a convergence point of many signal transduction pathways. NF-κB p50, NF-κB p65 and IκB are combined to form a trimer, and thus they all exist in a cell medium in an inactive form. When cells are exposed to external stimuli, IκB is phosphorylated and degraded. At this time, NF-κB is activated, enters the nucleus, and binds to the corresponding part of a target gene to start the transcription of related genes. NF-κB can induce the production of a plurality of cytokines and inflammatory factors, such as TNF-α and IL-1β.

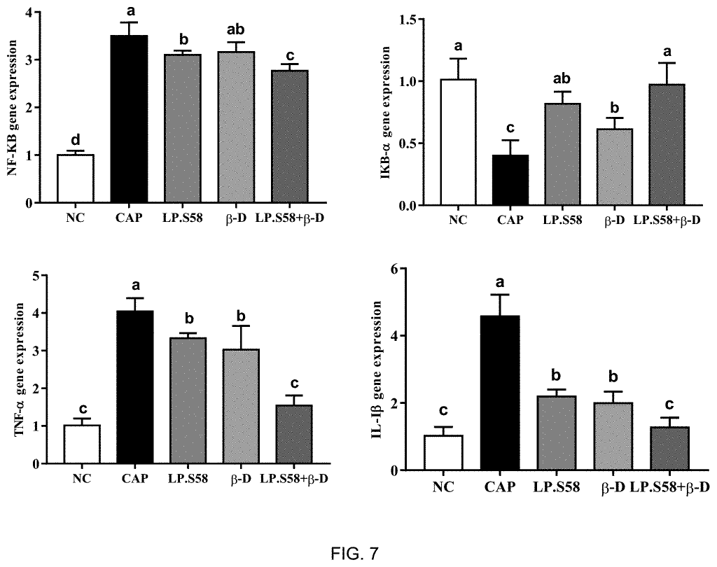

FIG. 7 illustrates the effects of LP.S58 and β-D on the expression of NF-κB, IκB-α, TNF-α, and IL-1β in the stomach. Compared with the NC group, expression levels of NF-κB, TNF-α, and IL-1β are up-regulated significantly and expression levels of IκB-α are down-regulated significantly in the CAP group; after intervention with LP.S58 or β-D alone, expression levels of NF-κB, TNF-α and IL-1β are partly down-regulated, and IκB-α is partly up-regulated, but administration of either one has an inferior effect to administration of both.

3.9 Effect of L. plantarum S58 Combined with Highland Barley β-Glucan on mRNA Expression of iNOS and eNOS in the Mouse Gastric Tissue

iNOS and eNOS are inducible and endothelial types of NOS, respectively. NOS is a rate-limiting enzyme for NO synthesis and widely exists in normal human and animal tissues. The expression and activity of eNOS are relatively stable. NO derived from eNOS is mainly involved in promoting mucosal epithelial repair, regulating gastric mucosal blood flow and adaptive cytoprotection, inhibiting gastric acid secretion, enhancing mucus barrier function and promoting vascular regeneration. Once iNOS is activated, enzyme activity will last for a long time, and a large amount of NO will be produced. Low concentrations of NO can effectively combat gene mutations and activate the body's defense capabilities, but high concentrations of NO will lose control of gene mutations, stimulate gene mutations, and induce tumors.

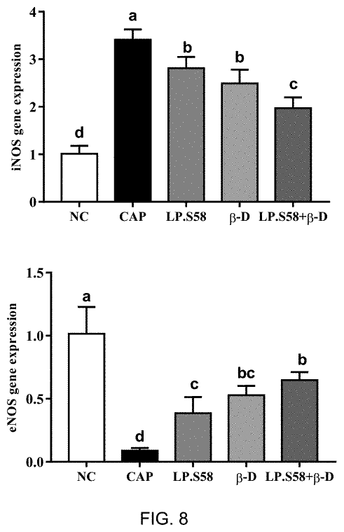

As can be seen from FIG. 8, except for the CAP group, the other four groups show low expression levels of iNOS and high expression levels of eNOS, with significant differences. This shows that LP.S58 and β-D can down-regulate the expression of iNOS and up-regulate the expression of eNOS, thereby inhibiting the inflammatory response, protecting the gastric mucosa and inhibiting gastric ulcer.

3.10 Effect of L. plantarum S58 Combined with Highland Barley β-Glucan on mRNA Expression of NF-κB, IκB-α, TNF-α and IL-1β in the Mouse Small Intestine

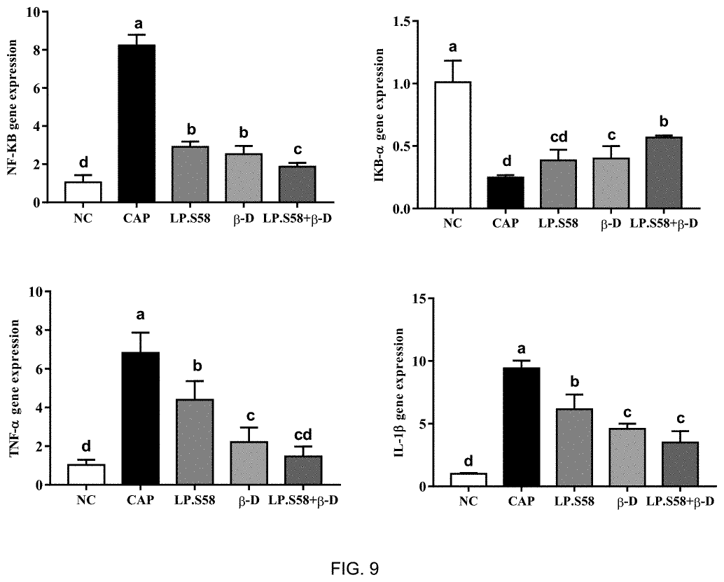

As can be seen from FIG. 9, administration of capsaicin by gavage can significantly up-regulate the expression levels of NF-κB, TNF-α and IL-1β genes in the small intestine tissue, and down-regulate the expression of IκB-α significantly; after intervention with LP.S58 and β-D, the expression levels of NF-κB, TNF-α and IL-1β are down-regulated significantly, the expression level of IκB-α is up-regulated significantly, and administration of both has a better effect. This shows that LP.S58 and β-D can alleviate the inflammatory damage to small intestine tissue caused by capsaicin.

3.11 Effect of L. plantarum S58 Combined with Highland Barley β-Glucan on mRNA Expression of ZO-1 and Occludin in the Mouse Small Intestine

ZO-1 and Occludin are important tight junction proteins in the intestine and play an important role in maintaining the integrity of the intestinal mucosal barrier of the small intestine tissue.

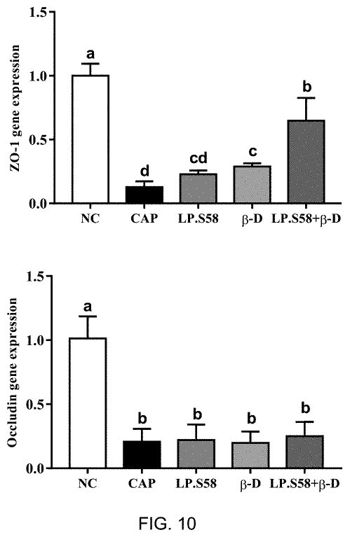

As can be seen from FIG. 10, after administration of capsaicin by gavage, expression levels of ZO-1 and Occludin are down-regulated significantly, indicating that capsaicin can destroy the intestinal mucosal barrier and change the intestinal permeability; administration of both LP.S58 and β-D can up-regulate the expression level of ZO-1 significantly, but then, their co-administration does not up-regulate the expression of Occludin.

Lastly, the above examples are only used to illustrate the technical solutions of the disclosure and not to limit them. Although the disclosure has been described by referring to the preferred examples of the disclosure, those of ordinary skill in the art should appreciate that various changes may be made in form and detail without departing from the spirit and scope of the disclosure as defined by the appended claims.

What is claimed is:

1.-3. (canceled)

4. A pharmaceutical composition for treating or preventing spicy food-induced damage to the digestive system, wherein the pharmaceutical composition comprises a pharmaceutically effective dose of Lactobacillus plantarum S58 with an accession number of CCTCC NO: M 2019595.

5. A food for alleviating spicy food-induced damage to the digestive system, wherein the food comprises Lactobacillus plantarum S58 with an accession number of CCTCC NO: M 2019595.

6. A health product for alleviating spicy food-induced damage to the digestive system, wherein the health product comprises Lactobacillus plantarum S58 with an accession number of CCTCC NO: M 2019595.

7. (canceled)


SEQUENCE LISTING (25 sequences)

SEQ ID NO: 1 - DNA, 1928 bp, Lactobacillus plantarum S58
gggatgcgcatgctatacatgcagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaaggaatctgtcacttaggcggctggttcctaaaaggttaccccaccgactttgggtgttacaaactctcatggtgtgacgggcggtgtgtacaaggcccgggaacgtattcaccgcggcatgctgatccgcgattactagcgattccgacttcatgtaggcgagttgcagcctacaatccgaactgagaatggctttaagagattagcttactctcgcgagttcgcaactcgttgtaccatccattgtagcacgtgtgtagcccaggtcataaggggcatgatgatttgacgtcatccccaccttcctccggtttgtcaccggcagtctcaccagagtgcccaacttaatgctggcaactgataataagggttgcgctcgttgcgggacttaacccaacatctcacgacacgagctgacgacaaccatgcaccacctgtatccatgtccccgaagggaacgtctaatctcttagatttgcatagtatgtcaagacctggtaaggttcttcgcgtagcttcgaattaaaccacatgctccaccgcttgtgcgggcccccgtcaattcctttgagtttcagccttgcggccgtactccccaggcggaatgcttaatgcgttagctgcagcactgaagggcggaaaccctccaacacttagcattcatcgtttacggtatggactaccagggtatctaatcctgtttgctacccatactttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaggctttcacatcagacttaaaaaaccgcctgcgctcgctttacgcccaataaatccggacaacgcttgccacctacgtattaccgcggctgctggcacgtagtta

SEQ ID NO: 2 - DNA, 21 bp, Artificial Sequence
  Primer, GAPDH-F
gaggtcaatgaaggggtcgtt

SEQ ID NO: 3 - DNA, 22 bp, Artificial Sequence
  Primer, GAPDH-R
ctcgtcccgtagacaaaatggt

SEQ ID NO: 4 - DNA, 18 bp, Artificial Sequence
  Primer, EGF-F
tgggtctcggattgggct

SEQ ID NO: 5 - DNA, 20 bp, Artificial Sequence
  Primer, EGF-R
accacaaccagtgacgaggg

SEQ ID NO: 6 - DNA, 21 bp, Artificial Sequence
  Primer, EGFR-F
gccatctgggccaaagatacc

SEQ ID NO: 7 - DNA, 22 bp, Artificial Sequence
  Primer, EGFR-R
gtcttcgcatgaataggccaat

SEQ ID NO: 8 - DNA, 21 bp, Artificial Sequence
  Primer, VEGF-F
tcgtccaacttctgggctctt

SEQ ID NO: 9 - DNA, 23 bp, Artificial Sequence
  Primer, VEGF-R
ccttctcttcctcccctctcttc

SEQ ID NO: 10 - DNA, 21 bp, Artificial Sequence
  Primer, NF-EB-F
atggcagacgatgatccctac

SEQ ID NO: 11 - DNA, 22 bp, Artificial Sequence
  Primer, NF-EB-R
cggaatcgaaatcccctctgtt

SEQ ID NO: 12 - DNA, 21 bp, Artificial Sequence
  Primer, IEB-A-F
tgaaggacgaggagtacgagc

SEQ ID NO: 13 - DNA, 19 bp, Artificial Sequence
  Primer, IEB-A-R
tgcaggaacgagtctccgt

SEQ ID NO: 14 - DNA, 19 bp, Artificial Sequence
  Primer, TNF-A-F
caggcggtgcctatgtctc

SEQ ID NO: 15 - DNA, 22 bp, Artificial Sequence
  Primer, TNF-A-R
cgatcaccccgaagttcagtag

SEQ ID NO: 16 - DNA, 22 bp, Artificial Sequence
  Primer, IL-1A-F
gaaatgccaccttttgacagtg

SEQ ID NO: 17 - DNA, 21 bp, Artificial Sequence
  Primer, IL-1A-R
tggatgctctcatcaggacag

SEQ ID NO: 18 - DNA, 23 bp, Artificial Sequence
  Primer, iNOS-F
gttctcagcccaacaatacaaga

SEQ ID NO: 19 - DNA, 19 bp, Artificial Sequence
  Primer, iNOS-R
gtggacgggtcgatgtcac

SEQ ID NO: 20 - DNA, 20 bp, Artificial Sequence
  Primer, eNOS-F
tcagccatcacagtgttccc

SEQ ID NO: 21 - DNA, 21 bp, Artificial Sequence
  Primer, eNOS-R
atagcccgcatagcgtatcag

SEQ ID NO: 22 - DNA, 19 bp, Artificial Sequence
  Primer, ZO-1-F
gccgctaagagcacagcaa

SEQ ID NO: 23 - DNA, 22 bp, Artificial Sequence
  Primer, ZO-1-R
gccctccttttaacacatcaga

SEQ ID NO: 24 - DNA, 22 bp, Artificial Sequence
  Primer, Occludin-F
tgaaagtccacctccttacaga

SEQ ID NO: 25 - DNA, 22 bp, Artificial Sequence
  Primer, Occludin-R
ccggataaaaagagtacgctgg